(19) United States
(12) Patent Application Publication
(10) Pub. No.: US 2005/0085528 A1
(43) Pub. Date: Apr. 21, 2005

(54) Parmaceutical

(75) Inventors: Tero Ahola, Sandwich (GB); Tim Jenkins, Sandwich (GB); Carl Laxton, Sandwich (GB); Ted Murray, Sandwich (GB); Tanya Parkinson, Sandwich (GB); Manoussos Perros, Sandwich (GB)

Correspondence Address: AGOURON PHARMACEUTICALS, INC., 10350 NORTH TORREY PINES ROAD, LA JOLLA, CA, 92037, US

(73) Assignee: Pfizer Inc.

(21) Appl. No.: 10/688,648

(22) Filed: Oct. 17, 2003

(51) Int. Cl. A61K 31/401; A61K 31/366; A61K 31/225

(52) U.S. Cl. 514/423; 514/460; 514/548

(57) ABSTRACT

Use of one or more prenylation inhibitors, cholesterol biosynthesis inhibitors or HMG-CoA reductase inhibitors in the manufacture of a medicament for the treatment of hepatitis C virus (HCV) infection. The invention also encompasses a pharmaceutical composition for use in the treatment of HCV infection, the pharmaceutical composition comprising one or more agents capable of inhibiting prenylation, cholesterol biosynthesis or HMG-CoA reductase in the liver, wherein the one or more agents is optionally admixed with a pharmaceutically acceptable carrier, diluent or excipient.

This application claims priority from United Kingdom application number 0224680.9, filed Oct. 23, 2002 and also claims the benefit of U.S. Provisional Application No. 60/432,789, filed Dec. 12, 2002, and incorporates each application by reference in its entirety.

### BACKGROUND OF THE INVENTION

The present invention relates to the treatment of hepatitis C, in particular, to the use of statins, such as atorvastatin and its analogues, in the treatment of hepatitis C.

The hepatitis C virus (HCV) is one of the most important causes of chronic liver disease. In the United States it accounts for about 15 percent of acute viral hepatitis, 60 to 70 percent of chronic hepatitis and up to 50 percent of cirrhosis, end-stage liver disease and liver cancer. Hepatitis C is the most common blood-borne infections in the United States, infecting more than 1.8 percent of the population and causing an estimated 8,000 to 10,000 deaths annually (National Institute of Health Consensus Development Conference Statement: Management of Hepatitis C: Jun. 10-12 (2002); http://www.consensus.nih.gov).

At least 75 percent of patients with acute Hepatitis C ultimately develop chronic infection and most of these patients have accompanying chronic liver disease. Chronic hepatitis C varies greatly in its course and outcome. At one end of the spectrum are patients who, apart from their viral load, have no signs or symptoms of liver disease and completely normal levels of serum liver enzymes. Liver biopsy usually shows some degree of damage, but the degree of injury is usually mild and the overall prognosis may be good. At the other end of the spectrum are patients with moderate to severe symptoms, high HCV RNA in serum and marked elevation of serum liver enzymes. These patients ultimately develop cirrhosis and end-stage liver disease. In the middle of the spectrum are many patients who have few or no symptoms, mild to moderate elevations in liver enzymes and an uncertain prognosis. Researchers estimate that at least 20 percent of patients with chronic hepatitis C develop cirrhosis, a process that can take 10 to 20 years. After 20 to 40 years, a smaller percentage of patients with chronic disease will develop liver cancer.

HCV is a small (50 to 60 nm in diameter), enveloped, positive, single-stranded RNA virus in the Flaviviridae family. The genome is approximately 10,000 nucleotides and encodes a single polyprotein of about 3,000 amino acids. The polyprotein is processed by host cell and viral proteases into three major structural proteins and several non-structural protein necessary for viral replication (Bartenschlager and Lohmann, J. Gen. Virol. (2002) 81, 1631-1648). Because the virus mutates rapidly, changes in the envelope protein are thought to contribute to evasion of the immune system. The virus displays extensive genetic heterogeneity: there are six known genotypes and more than 50 subtypes of hepatitis C, the different genotypes having different geographic distributions. Genotypes 1a and 1b are the most common in the United States while genotypes 2 and 3 are present in only 10 to 20 percent of patients. There is little difference in the severity of disease or outcome of patients infected with different genotypes. However, this genetic diversity impacts negatively on both treatment options and effectiveness. In particular, genotype 1, which accounts for >70 percent of all HCV infections in the US and has infected millions of people around the world via contaminated blood transfusions, is associated with the poorest response to treatment. Patients with genotypes 2 and 3 are more likely to respond to alpha interferon treatment (Zein, Clin. Microbiol. Reviews (2000) 13, 223-235).

Alpha interferon is a host protein that is made in response to viral infections and has natural antiviral activity. Recombinant forms of alpha interferon have been produced, and several formulations are available as therapy of hepatitis C. However, these standard forms of interferon are now being replaced by pegylated interferons (peginterferons). Peginterferon is alpha interferon that has been modified chemically by the addition of a large inert molecule of polyethylene glycol. Pegylation changes the uptake, distribution and excretion of interferon, prolonging its half-life, and therefore is more effective than standard interferon in inhibiting HCV, yielding higher sustained response rates with similar side effects.

Currently the most effective therapy appears to be a 24- or 48-week course of the combination of pegylated alpha interferon and ribavirin, an oral antiviral agent that has activity against a broad range of viruses. By itself, ribavirin has little effect on HCV, but adding it to interferon or peginterferon increases the sustained response rate by two- to three-fold. The optimal duration of treatment depends on viral genotype. Patients with genotypes 2 and 3 have a high rate of response to combination treatment (70 to 80 percent). In contrast, patients with genotype 1 have a lower rate of response to combination therapy (40 to 45 percent).

Alpha interferon has multiple neuropsychiatric effects and strict abstinence from alcohol is also recommended during therapy with interferon. Prolonged therapy can cause marked irritability, anxiety, personality changes, depression and even suicide or acute psychosis.

Alpha interferon therapy can induce auto-antibodies and a 6- to 12-month course triggers an autoimmune condition in about 2 percent of patients, particularly if they have an underlying susceptibility to autoimmunity. Exacerbation of a known autoimmune disease (such as rheumatoid arthritis or psoriasis) occurs commonly during interferon therapy. Alpha interferon also has bone marrow suppressive effects.

Ribavirin causes red cell haemolysis to a variable degree in almost all patients. Therefore, patients with a pre-existing haemolysis or anaemia should not receive ribavirin. Similarly, patients who have significant coronary or cerebral vascular disease should not receive ribavirin, as the anaemia caused by treatment can trigger significant ischaemia. Fatal myocardial infarctions and strokes have been reported during combination therapy with alpha interferon and ribavirin. Ribavirin is excreted largely by the kidneys and patients with renal disease can develop haemolysis that is severe and even life-threatening. Ribavirin also causes birth defects in animal studies, while alpha interferon has direct antigrowth and antiproliferative effects.

Few options exist for patients who either do not respond to therapy or who respond and later relapse. Patients who relapse after a course of interferon monotherapy may respond to a course of combination therapy, particularly if they became and remained HCV RNA negative during the period of monotherapy. Another approach is the use of long-term or continual interferon, which is feasible only if the interferon is well tolerated and has a clear-cut effect on serum aminotransferases and liver histology, despite lack of clearance of HCV RNA. Therefore, new medications and approaches to treatment are needed.

Statins represent a well-established class of drugs that effectively lower serum cholesterol levels and are widely prescribed for the treatment of hypercholesterolaemia. They can be grouped into naturally occurring compounds, such as lovastatin, pravastatin and mevastatin, and fully synthetic compound such as fluvastatin and atorvastatin. Simvastatin is a chemically modified form of lovastatin and therefore a second generation drug.

The synthetic statin atorvastatin, marketed as Lipitor® and disclosed in U.S. Pat. No. 5,273,995 which is incorporated herein by reference, is represented by the formula [R-(R*,R*)]-2-(4-fluorophenyl)-b,d-dihydroxy-5-(1-methylethyl)-3-phenyl-4[(phenylamino)carbonyl]-IH-pyrrole-1-heptanoic acid (C₃₃H₃₄FN₂O₅)₂Ca.3H₂O).

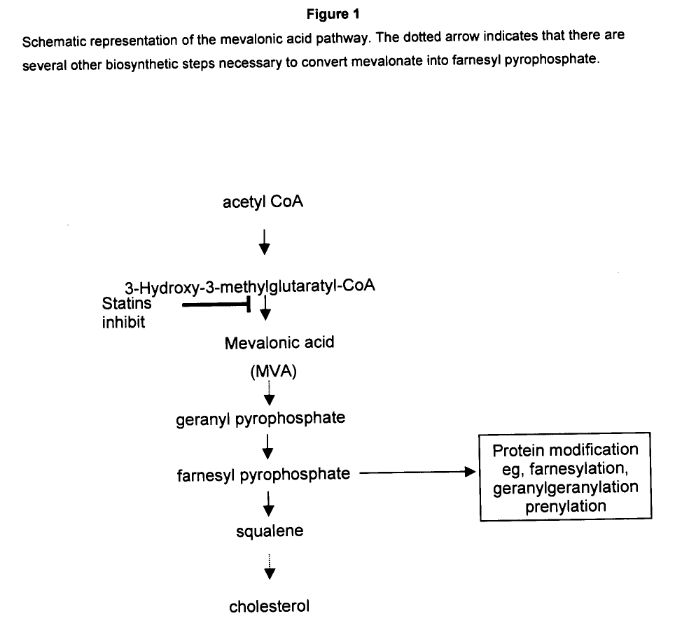

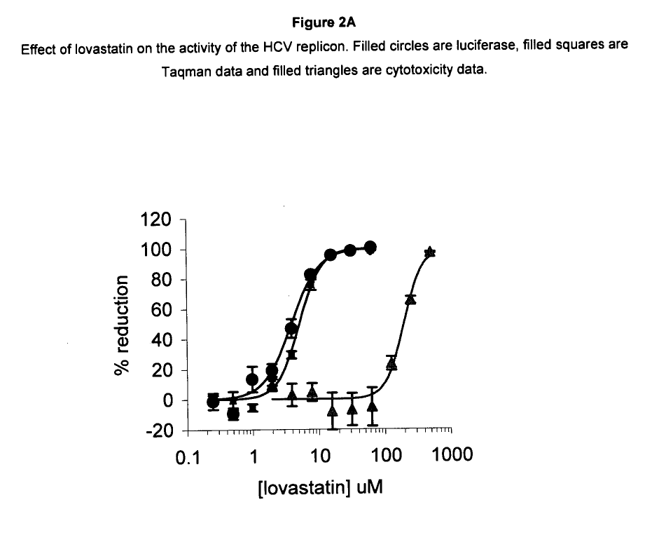

The conversion of 3-hydroxy-3-methylglutaryl-coenzyme A (HMG-CoA) to mevalonate is an early and rate-limiting step in the cholesterol biosynthetic pathway in the liver and other tissues. This step is catalyzed by the enzyme HMG-CoA reductase. Statins are competitive inhibitors of HMG-CoA reductase and, as such, are collectively potent lipid lowering agents. In addition to reducing cholesterol levels, the inhibition of HMG-CoA reductase may also reduce protein prenylation by lowering intracellular levels of isoprenoids, such as farnesyl- and geranylgeranyl pyrophosphate (FIG. 1).

Isoprenoids are necessary for the post-translational lipid modification (prenylation) of a variety of proteins, thereby anchoring them to the cell membrane (Zhang and Casey, Ann. Rev. Biochem. (1996) 65, 241-269). Statins block the conversion of HMG CoA to mevalonate, leading to reduced synthesis of cholesterol and decreased prenylation of proteins that play a key role in signal transduction pathways regulating cell proliferation, cell differentiation, vesicular transport and apoptosis (Zhang and Casey).

In addition to treating hypercholesterolaemia, statins are an effective therapy for other lipodystrophies. For example, hyperlipidaemia is known to occur in a significant percentage of patients taking potent protease inhibitors as part of anti-HIV therapy (reviewed in Fichtenbaum et al, AIDS (2002) 16, 569-577) and a recent randomised open-label study has shown that atorvastatin (or pravastatin) co-treatment is a well tolerated co-therapy to manage the hyperlipidaemia without adverse pharmacokinetic interactions with saquinavir or nelfinavir (Fichtenbaum et al).

It has recently been reported that lovastatin possesses antiviral activity in Respiratory Syncytial Virus (RSV) model systems in the micromolar range (Gower and Graham, AntiMicrobial Agents and Chemotherapy (2001) 45, 1231-1237; US 2002/0142940). The mode of action is believed to be inhibition of membrane localisation of functional proteins required for viral replication caused by the decreased level of isoprenyl moieties in the host cells. Similar antiviral modes of action have been reported for other prenylation inhibitors in Hepatitis Delta Virus (HDV) model systems (Glenn et al, J.Virol. (1998) 72, 9303-9306; Black et al, Arch. Inter. Med. (1998) 158, 577-584).

The use of agents that inhibit the prenylation or post-prenylation of viral proteins is disclosed in EP 0672192. In particular, it is shown that interference of the prenylation stage in the life cycle of HDV affects viral infection. The HDV genome is a single, negative-stranded RNA molecule and is the only animal virus known to have a circular RNA genome. HDV relies on hepatitis B virus (HBV) for transmission because it uses the hepatitis B surface antigen as its own virion coat.

WO 00/47196 discloses the use of anti-viral statin and statin-like compounds in the treatment of viral infections, such as HIV or hepatitis. The effects of simvastatin against bovine viral diarrhea virus (BVDV) and mevastatin, pravastatin, lovastatin, tovastatin and simvastatin against strains of HIV are exemplified. BVDV is a member of the genus Pestivirus within the flaviviridae family of virus and is spread between cattle via nasal and oral secretions, faeces and urine, spreading systemically through the blood stream both as free virus in the serum and virus infected leucocytes, particularly lymphocytes and monocytes. Symptoms of BVDV range from mild fevers, diarrhea and leukopenia to fatal haemorrhagic diarrhea and fatal thrombocytopenia.

### SUMMARY OF THE INVENTION

It is against this background that the present invention has been conceived. In its broadest sense, the invention encompasses the use of one or more prenylation inhibitors in the manufacture of a medicament for the treatment of hepatitis C virus (HCV) infection.

Preferably, the one or more prenylation inhibitors is a statin or statin-like compound. While statins are known inhibitors of prenylation, cholestasis and active liver disease are listed as contra-indications to statin use. However, no specific evidence exists showing exacerbation of liver disease by statins and progressive liver failure due to statins is exceedingly rare, if it ever occurs (Pedersen and Tobert, Drug Saf. (1996) 14, 11-24).

The term “statin-like compounds” encompasses analogues, derivatives, variants or mimetics of known statins. Examples of statins include lovastatin, mevastatin, pravastatin, simvastatin, fluvastatin, cerivastatin and atorvastatin.

Preferably, the statin is atorvastatin or an analogue, derivative, variant or mimetic thereof. Atorvastatin has an excellent and well documented safety profile, comparable to that of other statins, and is generally well-tolerated. Furthermore, atorvastatin shows superior effects overall when compared to other known statins in an in vitro model of HCV replication.

The prenylation inhibitor may be administered as monotherapy or separately, sequentially or simultaneously in combination with one or more anti-viral agents. Such anti-viral agents may be interferon and/or ribavirin. The combination of such compounds enhances the likelihood of substantially eliminating the virus from an individual.

From another aspect, the present invention relates to a pharmaceutical composition for use in the treatment of HCV infection, the pharmaceutical composition comprising one or more agents capable of inhibiting prenylation in the liver, wherein the one or more agents is optionally admixed with a pharmaceutically acceptable carrier, diluent or excipient.

Preferably, the one or more agent is a statin or statin-like compound, such as atorvastatin or an analogue, derivative, variant or mimetic thereof.

Advantageously, the pharmaceutical composition further comprises one or more anti-viral agents.

From a further aspect, the present invention relates to a method for treating an individual infected with HCV, the method comprising administering to the individual a therapeutically effective amount of one or more agents capable of inhibiting prenylation in the liver, wherein the one or more agents is optionally admixed with a pharmaceutically acceptable carrier, diluent or excipient.

The present invention also encompasses the use of one or more inhibitors of cholesterol biosynthesis in the manufacture of a medicament for the treatment of HCV infection.

From another aspect, the invention resides in a pharmaceutical composition for use in the treatment of HCV infection, the pharmaceutical composition comprising one or more agents capable of inhibiting cholesterol biosynthesis in the liver, wherein the one or more agents is optionally admixed with a pharmaceutically acceptable carrier, diluent or excipient.

From a further aspect, the invention relates to a method for treating an individual infected with HCV, the method comprising administering to the individual a therapeutically effective amount of one or more agents capable of inhibiting cholesterol biosynthesis in the liver, wherein the one or more agents is optionally admixed with a pharmaceutically acceptable carrier, diluent or excipient.

Preferably the one or more cholesterol biosynthesis inhibitors is a statin or statin-like compound, such as atorvastatin or an analogue, derivative, variant or mimetic thereof, which may be administered separately, sequentially or simultaneously in combination with one or more anti-viral agents. Such anti-viral agents may be interferon and/or ribavirin.

The present invention yet further encompasses the use of one or more inhibitors of HMG-CoA reductase in the manufacture of a medicament for the treatment HCV infection.

From another aspect, the invention resides in a pharmaceutical composition for use in the treatment of HCV infection, the pharmaceutical composition comprising one or more agents capable of inhibiting HMG-CoA reductase in the liver, wherein the one or more agents is optionally admixed with a pharmaceutically acceptable carrier, diluent or excipient.

From a further aspect, the present invention relates to a method for treating an individual infected with HCV, the method comprising administering to the individual a therapeutically effective amount of one or more agents capable of inhibiting HMG-CoA reductase in the liver, wherein the one or more agents is optionally admixed with a pharmaceutically acceptable carrier, diluent or excipient.

Preferably the one or more HMG-CoA reductase inhibitors is a statin or statin-like compound, such as astorvastatin or an analogue, derivative, variant or mimetic thereof, which may be administered separately, sequentially or simultaneously in combination with one or more anti-viral agents. Such anti-viral agents may be interferon and/or ribavirin.

The invention will now be described in detail with reference, but not limited, to the following figures in which:

FIG. 1 is a schematic representation of the mevalonic acid (MVA) pathway. The dotted arrow indicates that there are several other biosynthetic steps necessary to convert mevalonate into farnesyl pyrophosphate;

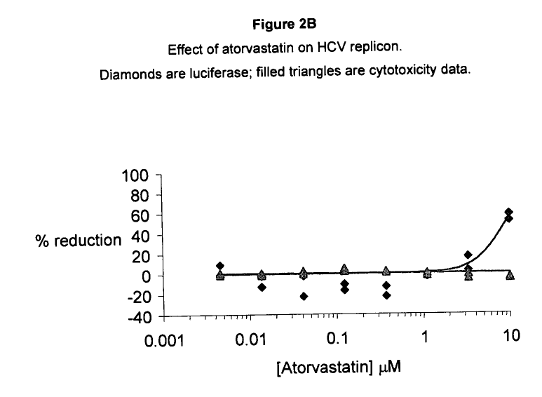

FIG. 2 in which FIG. 2a shows the effect of lovastatin on the activity of the HCV replicon. Filled circles are luciferase, filled squares are Taqman data and filled triangles are cytotoxicology data. FIG. 2b shows the effect of atorvastatin on HCV replicon;

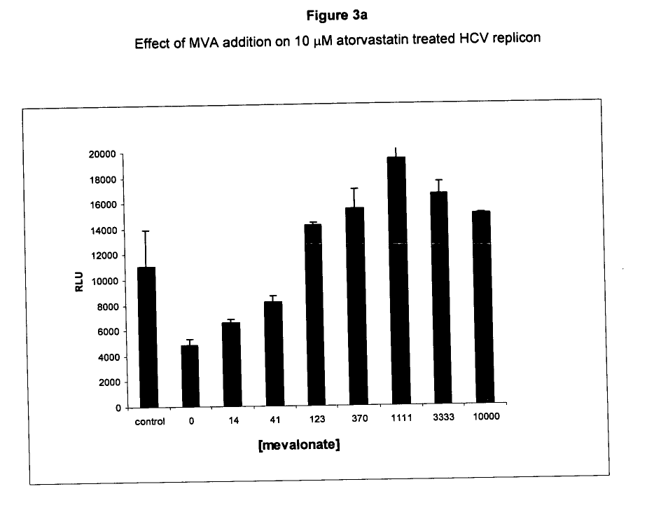

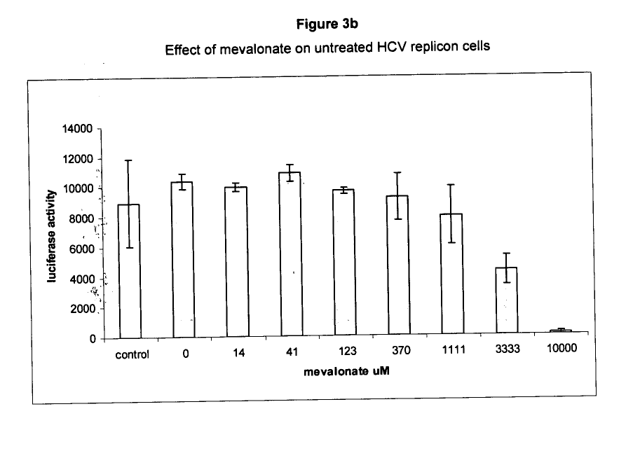

FIG. 3 in which FIG. 3a shows the effect of MVA addition on 10 uM atorvastatin treated HCV replicon. FIG. 3b shows the effect of mevalonate on untreated HCV replicon cells;

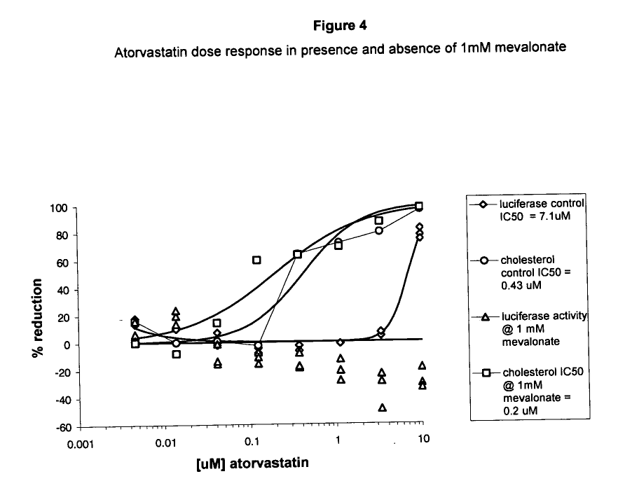

FIG. 4 shows a dose response for Atorvastatin in presence and absence of 1 mM mevalonate;

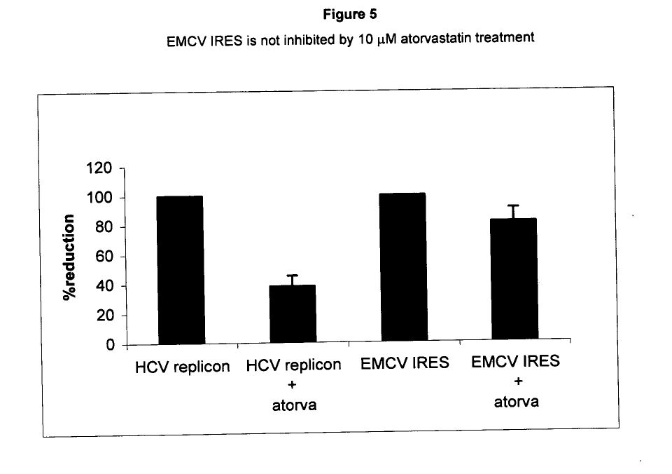

FIG. 5 shows the effect of 10 uM atorvastatin (atorva) on the Encephalomyocarditis virus internal ribosome entry site (EMCV IRES); and

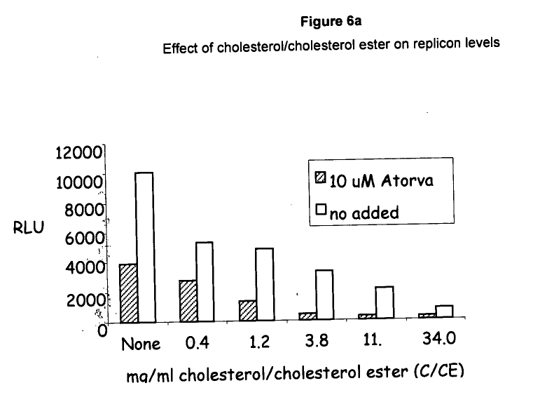

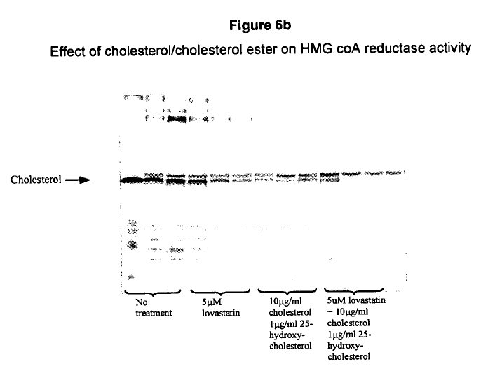

FIG. 6 in which FIG. 6a shows the effect of cholesterol/cholesterol ester on replicon levels and FIG. 6b shows the effect of cholesterol/cholesterol ester on HMG coA reductase activity.

### IN VITRO STUDIES

The HCV replicon (Huh 5-2 [I₃₈₉luc-ubi-neo-NS3-3′/5.1]) is an in vitro model of HCV replication in which the luciferase reporter is incorporated into HCV sequences (Lohmann et al, Science (1999) 285, 110-113; Krieger et al, J. Virol. (2001) 75, 4614-4624). The firefly luciferase reporter is expressed as a luciferase-ubiquitin-neomycin phosphotransferase fusion protein, which is cleaved by host proteases to release luciferase. The replicon also contains an internal EMCV IRES for translation of HCV NS3-5B polyprotein, which harbours cell culture adapted mutations to permit high cloning efficiency (Krieger et al). The luciferase output has been shown to be directly proportional to the level of HCV replicon RNA genomes present in the host cell, which can be directly measured by quantitative RT-PCR using the Taqman assay.

The HCV replicon system has been used to assay the antiviral effect of a range of statins and have generated quantitative IC₅₀ data for both efficacy and toxicity. Furthermore, results show that the statin-induced antiviral activity can be relieved by exogenous addition of mevalonic acid, (a metabolite downstream of the HMG CoA blockade).

Materials

DMEM complete medium: DMEM Glutamax (Life Technologies #31966-021) supplemented with 10% foetal calf serum, penicillin (100 IU/ml/ streptomycin (100 μg/ml) (Life Technologies #15140-114); DMEM passage medium: DMEM Glutamax (Life Technologies #31966-021) supplemented with 10% foetal calf serum, penicillin (100lU/ml/ streptomycin (100 μg/ml) (Life Technologies #15140-114) plus 500 μg/ml G418 (Geneticin) (Life technologies 10131-027); Luciferase Reagent Pack Promega E1501; Passive lysis buffer (Promega E1941); PBS (Life Technologies, #14040-091); Trypsin (Life Technologies T4424); WST-1 assay kit (Roche Biosciences); Beta actin normaliser control PCR primer/probe set (PE Biosystems 4310881); Qiagen RNAeasy RNA extraction kit; reverse transcription reagents kit ((PE Biosystems 8030234); Universal Master Mix (PE Biosystems 4304437)); BrdU uptake assay kit (Biotrak, RPN 250); cytomix buffer: 120 mM KCl, 0.15 mM CaCl₂, 10 mM K₂HP0₄/KH₂P0₄ (pH 7.6), 25 mM HEPES, 2 mM EGTA, 5 mM MgCl₂, 2 mM ATP and 5 mM glutathione—adjusted to pH 7.6 with KOH.

Statins

The lactone forms of Lovastatin (Calbiochem) and Simvastatin (Calbiochem) were hydrolysed by alkaline hydrolysis in 1M NaOH at 50° C. for 2 hours, followed by dilution in water. Atorvastatin (Pfizer), Pravastatin (Chempacific Corporation) and Cerivastatin (Sequoia Research Products Ltd.) were dissolved in water.

PCR Primers and Probes:

Taqman probes and primers were designed using Primer Express software (PE Biosystems) as outlined in appendix C of Taqman Universal PCR Master Mix protocol p/n 43044449 Rev B.

Probe sequence:
FAM 5′-CTCCACCCAAGCGGCCGGA- TAMRA
Forward PCR primer sequence:
5′ GATGGATTGCACGCAGGTT-3′
Reverse PCR primer sequence:
5′ GCCCAGTCATAGCCGAATAGC-3′

Statin Treatment for HCV Replicon Cultures

Replicon cells were passaged to maintain the cells at 50-90% subconfluence. Cells were then trypsinsed and resuspended at 5.55×10⁴ cells/ml in DMEM complete. Aliquots (180 μL) containing 10⁴ cells were added to a clear 96 well plate (for WST cytotoxic assay and RNA extraction) and a duplicate white/clear Wallac Isoplate (for luciferase assay). An additional clear 96 well plate was set up for BrdU uptake. The plates were incubated at 37° C., 5% CO₂ for 18 hours. For statin treatment, a 10× dilution series was generated in complete DMEM/10% DMSO in a 96 well plate in 9 three-fold steps from a 50 mM stock concentration. Statins (20 μL) were added to triplicate wells containing overnight seeded lucubineo cells and incubated for a further 72 hours at 37° C., 5% CO₂.

Luciferase Assay

The cells were washed with 200 μL PBS and all traces of PBS were removed before adding 25 μL Passive Lysis Buffer (Promega Lucifase assay kit E1501 as described in Tech bulletin 281). The cells were lysed for 30 minutes are room temperature before addition of 100 μL assay reagent. Light output was measured on a Victor luminometer and data stored for analysis.

Green Fluorescent Protein Assay

Replicon cells were electroporated in cytomix buffer at 10⁷ cells/mL at 960 uF, 270 V with varying amounts pIRES2-EGFP. Cell viability was monitored by trypan blue staining and cell counting. Aliquots of cells (2×10⁴) were plated out in clear bottomed black 96 well plates and incubated for 24 hours. The cells were rinsed with PBS and fluorescence output read using an Analyst HT (LJL Biosystems).

Cytotoxity Assay and Quantitative RT-PCR

Statin-treated cells in the duplicate clear plate were analysed for cytotoxicity by addition of 10 μL WST-1 reagent (Roche Biosciences) to each well and incubated for 37° C. for 60 minutes. After vigorous shaking, the plate was read at 450 nm. The supernatent was removed and the monolayer washed with PBS prior to addition 100 μL RLT lysis buffer (Qiagen RNAeasy kit) to each test well, followed by vigorous resuspension to ensure uniform lysis of all cells. Total RNA was extracted according to Qiagen RNAeasy protocols recommended by manufacturer. Briefly, 0.1 μg total RNA was reverse transcribed using random hexamers and 20% cDNA produced was used as a template in a Taqman reaction (Universal PCR Master Mix protocol p\n 43044449 Rev B).

Cytostaticity Assay (BrdU Uptake Assay)

The Biotrak RPN 250 assay kit was used to perform this assay. On day three, 20 μL BrdU stock solution (100 μM,) was added to the wells of the third 96 well plate and cells were cultured for 2 hours. The supernatent was removed and 200 μL fixative was added prior to incubation with peroxidase labelled anti-BrdU and further developed according to manufacturers instructions.

Luciferase Data Analysis

Raw data from the Bertholdt Victor Multilabel plate reader was loaded into Microsoft Excel. Treated test wells and untreated control were corrected for background by substracting end-row well blanks (medium only, no cells) for each well (tests and controls). The percent reduction of each drug treated well was calculated by:

$$1 - ( \frac{Drug treated RLU}{Control RLU} ) \times 100$$

where RLU stands for relative luciferase units. The triplicate % reduction values (excluding untreated values) were plotted using multiple curve-fit in Labstats.
RT-PCR Data Analysis

These procedures are described in Perkin Elmer Biosystems User Bulletin 2. Briefly, the quantity of replicon RNA in a given sample was determined relative to the untreated control and normalised with respect to actin. Having established that the efficiencies of the replicon and actin PCR reactions were similar (data not shown), the relative quantity of replicon RNA was described by 2^{-ΔΔCt} where,
ΔΔCt=ΔCt₍ₜₑₛₜ₎−ΔCt_{(control)}
ΔCt₍ₜₑₛₜ₎−Ct_{(drug treated)}−Ct_{(actin)}
and
ΔCt_{(control)}=Ct_{(control)}−Ct_{(actin)}
Cholesterol Biosynthesis Assay

Replicon cells were plated at a density of 2×10⁵ per well in 450 μL complete medium in 24 well plates. The wells were treated with statins as described above. After 72 hours in culture the 24 well plates were taken out of the CO₂ incubator and 20 μL ¹⁴C acetate (activity1 mCi/5 mL) was added to each well. The plates were sealed with parafilm to avoid evaporation and incubated at 37° C. for 6 hrs on an orbital shaker at 25 rpm. The samples were saponified; by adding 1 mL 5M KOH (in 100% MeOH) to each well, resealing, incubating at 70° C. for 2 hrs and then overnight at room temperature. After the saponification was completed the media plus cells from the wells was transferred to a 15 mL glass round bottom tube and extracted with 4.0 mL hexane by vortexing for 10 sec. and separating the organic phase (top) into a clean glass tube. This extraction was repeated once more. Both organic phases of the same sample were pooled and dried under nitrogen. 1 mL hexane was added to the dry lipids vortexed well for 10 seconds and dried under nitrogen.

Following extraction, the dry samples were resuspended in chloroform (30 μL) by vigorous vortexing for 10 seconds and spotted onto 60A silica gel TLC plates. ¹⁴C cholesterol (0.1 μCi) was spotted onto an empty lane as a marker. The plates were developed in a solvent system of hexane:diethylether:acetic acid (70:30:2). The plates were developed until the solvent front was within 0.5 cm of the top of the TLC plate (approx 1 hr). After the chromatography was completed the plates were air dried in a fume hood for 1 hour at room temperature.

The TLC plates were exposed to a phospho-screeen for 24 hr. A Molecular Dynamics ‘STORM’ analyzer was used to scan the screens. The scanned image was then visually inspected and the cholesterol bands located according to the position of the ¹⁴C cholesterol marker. A volume report (using average background correction) for all the cholesterol bands was generated using the Imagequant 5.0 analysis software molecular dynamics and exported as an Excel data file. Percentage reduction values were calculated for each sample relative to the mean of the untreated controls using the formula (1-(vol.sample/mean vol control))*100. Data was plotted on a bar chart.

Results

Effect of HMG CoA Reductase Inhibition on Replicon Activity

The effect of modulating cholesterol biosynthesis on HCV replicon activity was studied. FIG. 2A shows a dose dependent inhibition of luciferase expression in replicon cells by lovastatin. The inhibition of replicon activity was confirmed by RT-PCR and showed lovastatin induced a quantitative reduction of replicon RNA (FIG. 2A). The RT-PCR data was normalised to beta actin RNA which remained constant throughout the experiment. Lovastatin is known to cause cell cycle arrest in transformed cells at G1 in a reversible manner (Jaobisiak et al, Proc. Natl. Acad. Sci. (1991) 88, 3628-3632) and it has been reported that replicon RNA levels fall in quiescent host cells (Pietschmann et al, J. Virol. (2001) 75, 1252-1264). Therefore, it was important to ensure that the down regulation of the replicon observed in our experiments was not due to indirect cell cycle effects. Thus, a BrdU uptake assay was used to measure DNA synthesis during lovastatin treatment. Table 1 shows that cell cycle cytostatic effect of lovastatin (IC₅₀ 89.3 μM±14.7) is far above the efficacious antiviral dose (IC₅₀ 7.9 μM). Overt toxicity is not observed until very high doses are attained (IC₅₀ 157.5 μM; see Table 1). Other HMG CoA reductase inhibitors were assayed in the HCV replicon system, including atorvastatin, and FIG. 2B shows that atorvastatin not only inhibits the replicon, but exhibits increased antiviral activity compared to lovastatin (see Table 1). The ability of each statin to reduce cholesterol levels in replicon cells was also measured.

TABLE 1
Antiviral activities of a range of statins compared against the
respective cholesterol inhibition activities.

| Statin | Replicon IC₅₀ (μM) | Cholesterol IC₅₀ (μM) | Cytotoxicity (μM) | Cytostatic (μM) |
|---|---|---|---|---|
| Atorvastatin | 4.1 +/− 1.6 | 0.6 +/− 0.1 | 18.3% @ 10 μM | 2.5 |
| Lovastatin | 7.9 +/− 1.1 | 3.3 +/− 1.1 | 157.5 +/− 33.1 | 89.3 +/− 14.7 |
| Simvastatin | 6.5 +/− 1.1 | 1 | 64.7 +/− 18.1 | 28.0 +/− 11.0 |
| Cerivastatin | 1.1 +/− 0.4 | 0.1 | 4.9 +/− 1.2 | 5.5 +/− 2.5 |
| Pravastatin | 322 | 17.5 +/− 6.4 | 0% @ 500,000 μM | 25% @ 500,000 μM |

The antiviral activities of the statins appeared to rank with their potencies for HMG CoA inhibition. Table 1 shows that the most potent cholesterol biosynthesis inhibitor (cerivastatin) also exhibited the most potent antiviral activity. The converse was true for pravastatin, which was the least active cholesterol biosynthesis inhibitor and also corresponded to the weakest antiviral activity. Interestingly, the cholesterol biosynthesis potency was usually 5-10 fold more potent than the antiviral activity, suggesting that cholesterol biosynthesis should be significantly inhibited.

Rescue of Statin Induced Antiviral Activity by Addition of Mevalonic Acid

FIG. 1 shows a schematic of the cholesterol biosynthesis pathway. Statins block HMG CoA reductase activity and therefore inhibit the formation of mevalonic acid. If the statin antiviral activity was due to HMG CoA reductase inhibition, then it should be possible to rescue the replicon by circumventing this block via addition of exogenous mevalonic acid. FIG. 3A shows that addition of 14 μM MVA begins to alleviate the atorvastatin inhibition of HCV replicon activity, as expected if the antiviral MoA stems from HMG CoA inhibition. Similar data was obtained for lovastatin treated replicon cells (data not shown).

Further addition of MVA not only completely restores replicon activity in the presence of 10 μM atorvastatin, but can result in replicon activity above the control level. Importantly, FIG. 3B shows that control HCV replicon cells do not respond to MVA in the absence of statin, therefore the MVA induced replicon activity represents a rescue of statin induced inhibition. These findings were further confirmed by carrying out an atorvastatin dose response experiment in the presence of 1 mM mevalonate (FIG. 4).

Statins Do Not Inhibit EMCV IRES Activity

The HCV replicon contains an encephalomyocarditis virus internal ribosome entry site (EMCV IRES) which drives expression of the HCV polyprotein. Impairment of this function was examined in the presence of statins. Replicon cells were transfected with varying amounts of pIRES2-EGFP in which the green fluorescent protein reporter is expressed by a EMCV IRES. FIG. 5 shows that atorvastatin treatment had no effect on the GFP signal at concentrations which reduced HCV replicon activity within the same cells. This suggests that the EMCV IRES is not being targeted by statins in the HCV replicon.

Effect of Cholesterol on Statin Inhibition of Replicon

Various amounts of a 10:1 mixture of cholesterol:25-hydroxy cholesterol ester were added to replicon-containing cells either in the presence or absence of atorvastatin.

FIG. 6a) shows that addition of cholesterol/cholesterol ester in the absence of statin reduced replicon levels. In the presence of statin, there was an even greater reduction in replicon levels. This is likely due to exogenously added cholesterol down-regulating HMG coA reductase activity. This was confirmed by measuring the amount of cholesterol synthesised.

FIG. 6b) shows that lovastatin and cholesterol/cholesterol ester when added individually reduce cholesterol levels, but when added together there is an even greater reduction in cholesterol levels. Thus addition of cholesterol/cholesterol ester causes a down-regulation of HMG coA reductase and when combined with HMG coA reductase inhibition by statin, this gives an even greater inhibitory effect on the HCV replicon.

A notable point from this experiment is that, despite addition of larg amounts of exogenous cholesterol, the effects of HMG coA reductase inhibition on replicon levels could not be reversed. This may suggest that inhibition of the HCV replicon occurs as a result of inhibition of the prenylation branch of the pathway.

Discussion

The results show that statins exhibit an antiviral activity mediated through HMG CoA reductase inhibition as shown by MVA addition. Furthermore, the results show that the statin-induced antiviral activity ranks with the relative potencies of sterol synthesis inhibition in replicon cells. Several groups have assayed lovastatin-mediated inhibition of sterol synthesis in Hep G2 cells (another hepatoma cell line) and all have reported similar activities (IC₅₀=51 nM (Bischoff et al, Atherosclerosis (1998) 139 suppl 1:S7-13); IC₅₀=10 nM (Bischoff et al, Atherosclerosis (1997) 135, 119-130); IC₅₀=24 nM (Cohen et al, Biochem. Pharmacol. (1993) 46, 1101)). Similar IC₅₀ values have been reported in lovastatin treated HeLa and CHO-KI cells (Sinensky et al, J. Biol. Chem. (1990) 265, 19937-19941). In contrast, the results presented here show that the sterol synthesis activity is very much higher in replicon cells (IC₅₀=3.3 μM±1.1). Such a discrepancy may be due to the long time frame over which the present assays were carried out (3 days), which may allow compensatory induction of cholesterol biosynthesis enzymes.

Protocol for Clinical Trial

Results obtained in cell culture, using a subgenomic replicon of HCV, clearly indicate that statins can inhibit HCV replication. Statins are known to concentrate in the liver of patients treated for hypercholesterolemia and the liver is believed to be the main site of HCV replication. Indeed, continual infection of hepatocytes appears to maintain this chronic infection. A clinically efficacious dose of a statin, such as atorvastatin, or statin-like compound should lead to a dramatic reduction in viral load as detected in patient serum.

Therefore, the aim of this study is to establish that the inhibition of HCV replication observed in vitro is mirrored by the ability of statins, such as atorvastatin, or statin-like compounds to reduce HCV viral load in chronically infected patients.

Patients chronically infected with HCV are administered with up to 40 mg o.d., atorvastatin in a double blind placebo-controlled study over 21 days to investigate the ability of statins to decrease viral load.

40 mg/day is currently an FDA approved dose for the treatment of hypercholesterolemia. Extensive clinical trials with atorvastatin indicate that at 40 mg/day, within the first 16 weeks of treatment, only 0.6% of patients experience elevation in liver transaminases levels greater than three times the upper limit of normal. In these same clinical trials patients receiving placebo experienced a 0.4% incidence of elevations in liver transaminases levels. Only at higher levels of atorvastatin are greater transaminases elevations seen. For example, at 80 mg/day 2.3% of patients had transaminases levels greater than three times the upper limit during the first 16 weeks (Black et al, Arch. Inter. Med. (1998) 158, 577-584).

Subjects of the study are HCV infected males or females, between the ages of 18-35 who display a stable levels of HCV infection, i.e. viral load, and who have no evidence of liver disease. Subjects are randomly assigned (20-25 per group) to either a placebo or treatment group.

1. A method for the treatment of hepatitis C infection comprising the step of administering to a subject in need thereof an effective amount of a prenylation inhibitor or a pharmaceutically acceptable salt thereof.

2. A method according to claim 1, wherein said prenylation inhibitor is a statin or statin-like compound.

3. A method according to claim 2, wherein said statin is atorvastatin or an analogue, derivative, variant or mimetic thereof.

4. A method according to any one of claims 1 to 3, wherein said prenylation inhibitor is administered separately, sequentially or simultaneously in combination with one or more anti-viral agents.

5. A pharmaceutical composition for use in the treatment of HCV infection, said pharmaceutical composition comprising one or more agents capable of inhibiting prenylation in the liver, wherein said one or more agents is optionally admixed with a pharmaceutically acceptable carrier, diluent or excipient.

6. The pharmaceutical composition according to claim 5, wherein said one or more agents is a statin or statin-like compound.

7. The pharmaceutical composition according to claim 6, wherein said statin is atorvastatin or an analogue, derivative, variant or mimetic thereof.

8. The pharmaceutical composition according to any one of claims 5 to 7, wherein said pharmaceutical composition further comprises one or more anti-viral agents.

9. A method for treating an individual infected with HCV comprising administering to said individual a therapeutically effective amount of one or more agents capable of inhibiting prenylation in the liver, wherein said one or more agents is optionally admixed with a pharmaceutically acceptable carrier, diluent or excipient.

10. The method according to claim 9, wherein said one or more agents is a statin or statin-like compound.

11. The method according to claim 10, wherein said statin is atorvastatin or an analogue, derivative, variant or mimetic thereof.

12. The method according to any one of claims 9 to 11, wherein said method further comprises separate, sequential or simultaneous administration of one or more anti-viral agents.

13. A method for the treatment of hepatitis C infection comprising the step of administering to a subject in need thereof an effective amount of a cholesterol biosynthesis inhibitor.

14. A method according to claim 13, wherein said cholesterol biosynthesis inhibitor is a statin or statin-like compound.

15. A method according to claim 14, wherein said statin is atorvastatin or an analogue, derivative, variant or mimetic thereof.

16. A method according to any one of claims 13 to 15, wherein said cholesterol biosynthesis inhibitor is administered separately, sequentially or simultaneously in combination with one or more anti-viral agents.

17. A pharmaceutical composition for use in the treatment of HCV infection, the pharmaceutical composition comprising one or more agents capable of inhibiting cholesterol biosynthesis in the liver, wherein the one or more agents is optionally admixed with a pharmaceutically acceptable carrier, diluent or excipient.

18. The pharmaceutical composition according to claim 17, wherein the one or more agents is a statin or statin-like compound.

19. The pharmaceutical composition according to claim 18, wherein the statin is atorvastatin or an analogue, derivative, variant or mimetic thereof.

20. The pharmaceutical composition according to any one of claims 17 to 19, wherein the pharmaceutical composition further comprises one or more anti-viral agents.

21. A method for treating an individual infected with HCV, the method comprising administering to the individual a therapeutically effective amount of one or more agents capable of inhibiting cholesterol biosynthesis in the liver, wherein the one or more agents is optionally admixed with a pharmaceutically acceptable carrier, diluent or excipient.

22. The method according to claim 21, wherein the one or more agents is a statin or statin-like compound.

23. The method according to claim 22, wherein the statin is atorvastatin or an analogue, derivative, variant or mimetic thereof.

24. The method according to any one of claims 21 to 23, wherein the method further comprises separate, sequential or simultaneous administration of one or more anti-viral agents.

25. A method for the treatment of HCV infection comprising the step of administering to a subject in need thereof an effective amount of an inhibitor of HMG-CoA reductase.

26. A method according to claim 25, wherein said HMG-CoA reductase inhibitor is a statin or statin-like compound.

27. A method according to claim 26, wherein said statin is atorvastatin or an analogue, derivative, variant or mimetic thereof.

28. A method according to any one of claims 25 to 27, wherein said HMG-CoA reductase inhibitor is administered separately, sequentially or simultaneously in combination with one or more anti-viral agents.

29. A pharmaceutical composition for use in the treatment of HCV infection, the pharmaceutical composition comprising one or more agents capable of inhibiting HMG-CoA reductase in the liver, wherein the one or more agents is optionally admixed with a pharmaceutically acceptable carrier, diluent or excipient.

30. The pharmaceutical composition according to claim 29, wherein the one or more agents is a statin or statin-like compound.

31. The pharmaceutical composition according to claim 30, wherein the statin is atorvastatin or an analogue, derivative, variant or mimetic thereof.

32. The pharmaceutical composition according to any one of claims 29 to 31, wherein the pharmaceutical composition further comprises one or more anti-viral agents.

33. A method for treating an individual infected with HCV, the method comprising administering to the individual a therapeutically effective amount of one or more agents capable of inhibiting HMG-CoA reductase in the liver, wherein the one or more agents is optionally admixed with a pharmaceutically acceptable carrier, diluent or excipient.

34. The method according to claim 33, wherein the one or more agents is a statin or statin-like compound.

35. The method according to claim 34, wherein the statin is atorvastatin or an analogue, derivative, variant or mimetic thereof.

36. The method according to any one of claims 33 to 35, wherein the method further comprises separate, sequential or simultaneous administration of one or more anti-viral agents.
